(19) United States
(12) Patent Application Publication
(10) Pub. No.: US 2024/0341796 A1
(43) Pub. Date: Oct. 17, 2024

(54) ENDOVASCULAR METHOD FOR BYPASSING AN OCCLUSION

(71) Applicant: Mauro Carlino, Milan (IT)

(72) Inventor: Mauro Carlino, Milan (IT)

(21) Appl. No.: 18/750,536

(22) Filed: Jun. 21, 2024

Related U.S. Application Data
(60) Provisional application No. 63/527,548, filed on Jul. 18, 2023.

(30) Foreign Application Priority Data
Mar. 7, 2023 (WO) PCT/EP2023/055752

(51) Int. Cl. A61B 17/32; A61B 17/22; A61M 25/01; A61M 25/10

(52) U.S. Cl. CPC A61B 17/320016; A61B 2017/22038; A61B 2017/22061; A61B 2017/22095; A61B 2017/320048; A61B 2017/320056; A61M 2025/0166; A61M 2025/1043

(57) ABSTRACT

An endovascular method for bypassing an occlusion is disclosed. A distal end of a guidewire may be advanced through a microcatheter and into a subintimal space of an artery of a patient, where a knuckle may be formed at the distal end of the guidewire, and the guidewire with the knuckle at the distal end thereof may be advanced to the occlusion. An inflatable balloon catheter may be placed over the advanced guidewire and distal to the occlusion, and the inflatable balloon catheter may be inflated to form at least one opening in a layer separating the subintimal space from a true lumen, whereby the guidewire may traverse the at least one opening.

### CROSS-REFERENCES TO RELATED APPLICATIONS

This application claims the benefit of U.S. Provisional Patent Application No. 63/527,548, filed Jul. 18, 2023, Patent Cooperation Treaty Patent Application No. PCT/EP2023/055752 filed Mar. 8, 2022, and Italian Patent Application No. 202200004412 filed Mar. 9, 2022 all of which are hereby incorporated by reference.

A portion of the disclosure of this patent document contains material that is subject to copyright protection. The copyright owner has no objection to the reproduction of the patent document or the patent disclosure, as it appears in the U.S. Patent and Trademark Office (USPTO) patent file or records, but otherwise reserves all copyright rights whatsoever.

### FIELD OF THE DISCLOSURE

The present disclosure relates to an endovascular method for bypassing an occlusion.

### BACKGROUND

Endovascular intervention techniques are understood to restore the normal physiological conditions of blood vessels, commonly arteries and coronary arteries, in patients with conditions affecting the blood vessels. One example of a condition affecting patients is a progressive reduction of a blood vessel lumen for the passage of blood flow, often caused by deposits of fat and plaque. The buildup of plaque may result in an abnormal narrowing or stricture of the blood vessel, otherwise known as stenosis, and may progress to an obstruction or closure of the vessel resulting in a complete or nearly complete blockage of the vessel, otherwise known as an occlusion. If left unchecked, the buildup of plaque within a vessel may lead to a chronic total occlusion (CTO), which is a complete blockage of the blood vessel typically lasting for three (3) months or longer. In the past, treatment options for patients suffering from symptoms of occlusions were limited to medication or coronary artery bypass grafts (CABG)—an open-heart surgery in which a vein or artery is taken from another part of the body and used to create a new path for blood to flow. More recently, techniques commonly known as percutaneous coronary intervention (PCI) provide a non-surgical procedures that utilizes a catheter to space a small structure called a stent to open up blood vessels that have been narrowed or blocked by plaque buildup.

Current treatment methods for percutaneous coronary intervention (PCI) may employ subintimal dissection and re-entry techniques to cross an occlusion and include techniques known as Subintimal Tracking And Re-entry (STAR), mini-STAR, contrast-guided STAR, Limited Antegrade Subintimal Tracking (LAST), Stingray, Controlled Antegrade and Retrograde Tracking (CART), and Reverse CART. Moreover, current antegrade dissection and re-entry (ADR) techniques involve crossing an occlusion with a guidewire or other equipment inserted into the subintimal or extra-plaque space (EPS) of a vessel, which is then followed by guidewire re-entry into a distal true lumen of the vessel. This guidewire distal true lumen re-entry may or may not be assisted by a dedicated device for directed in the guidewire into the distal true lumen. Once distal true lumen communication has been restored, inflation of a balloon and/or deploying a stent may take place.

Current antegrade dissection and re-entry (ADR) techniques, such as the Subintimal Tracking And Re-entry (STAR) technique, fail to provide a predictable location for re-entry of the guidewire to the distal true lumen. Current antegrade dissection and re-entry (ADR) techniques also result in a subintimal or extra-plaque space (EPS) dissection that are larger than necessary and may often result in an intra-extra-plaque space (EPS) hematoma. The result associated with these intra-extra-plaque space (EPS) hematomas is the permanent occlusion of side branch vessels and a reduced final Thrombolysis in Myocardial Infarction (TIMI) flow grade, thereby negatively affecting patient outcome. Additional antegrade dissection and re-entry (ADR) techniques, such as the contrast-guided STAR and Mini-STAR techniques, fail to reduce the likelihood of or otherwise improve the condition of extra-plaque space (EPS) hematomas.

One evolution of the original STAR technique has been the concept of “deferred stenting.” Suboptimal angiographic flow grade after stenting may be the primary driver of restenosis after successful STAR. In some instances, if stenting was not performed immediately, 70% of patients maintained TIMI 3 flow at 3-month angiographic follow-up, with the frequent reappearance of side branches originating from the dissected segment.

Other current methods in antegrade dissection and re-entry (ADR) techniques, known as antegrade fenestration and re-entry (AFR), improve upon the location for true lumen re-entry as compared to the Subintimal Tracking And Re-entry (STAR), mini-STAR, and contrast-guided STAR techniques, but lack confidence and predictability in ensuring a wire actually crosses from the subintimal or extra-plaque space (EPS) into the distal true lumen of a vessel. The antegrade fenestration and re-entry (AFR) technique involves ballooning within the subintimal or extra-plaque space (EPS) at the level of the distal cap of an occlusion to create transient fenestrations in the wall separating the subintimal or extra-plaque space (EPS) from the true lumen. Current antegrade dissection and re-entry (ADR) techniques, and in particular the antegrade fenestration and re-entry (AFR) technique due to the transient nature of the fenestrations created by AFR, have multiple shortcomings, which include failure to ensure the guidewire quickly gains access to the distal true lumen of the vessel through engagement of openings in the wall between the subintimal or extra-plaque space (EPS) and the true lumen, ineffective treatment of the subintimal hematoma, and failure to restore flow from side branches along the length of the occlusion

### BRIEF SUMMARY

Embodiments include a novel endovascular method for bypassing an occlusion.

Embodiments of methods and systems further optionally provide a solution to the shortcomings above. In some aspects, the techniques described herein relate to an endovascular method for bypassing an occlusion, including the steps of: (a) advancing a distal end of a guidewire through a microcatheter and into a subintimal space of an artery of a patient; (b) forming a knuckle at the distal end of the guidewire; (c) advancing the guidewire with the knuckle at the distal end to the occlusion; (d) placing an inflatable balloon catheter over the advanced guidewire distal to the occlusion; (e) forming at least one opening in a layer separating the subintimal space from a true lumen by inflating the inflatable balloon catheter; and (f) traversing the at least one opening with the guidewire.

In some aspects, the techniques described herein relate to a method, further including the step of: between steps e) and f), deflating the balloon catheter before traversing the at least one opening with the guidewire.

In some aspects, the techniques described herein relate to a method, wherein forming the knuckle of step b) further includes the distal end of the guidewire not forming a closed loop.

In some aspects, the techniques described herein relate to a method, wherein step c) further includes advancing the knuckle no further distal to the occlusion than is required to place a distal portion of the balloon catheter across a distal end of the occlusion.

In some aspects, the techniques described herein relate to a method, further including the step of: between steps c) and d), confirming accurate placement of the guidewire for targeted reentry by injecting a contrast media into the subintimal space.

In some aspects, the techniques described herein relate to a method, wherein forming the at least one opening of step e) further includes inflating the balloon catheter at least two times.

In some aspects, the techniques described herein relate to a method wherein: step d) further includes placing the inflatable balloon catheter over the advanced guidewire along an entire length of the occlusion; and forming at least one opening of step e) further includes inflating the balloon catheter along the entire length of the occlusion.

In some aspects, the techniques described herein relate to a method, further including the step of: between steps e) and f), retracting the balloon catheter and the guidewire.

In some aspects, the techniques described herein relate to a method, wherein: the distal end of the guidewire is maintained distal to a distal end of the balloon catheter.

In some aspects, the techniques described herein relate to a method, further including the step of: after step f), injecting a contrast media into the subintimal space through the microcatheter.

In some aspects, the techniques described herein relate to a method, further including the step of: after step f), performing one or more inflations of the balloon catheter in a position distal to proximal of the occlusion.

In some aspects, the techniques described herein relate to a method, further including the step of: after step f), exchanging, through the microcatheter, the guidewire with a workhorse guidewire.

In some aspects, the techniques described herein relate to a method, further including the step of: after step f), removing the balloon catheter; and placing a stent in at least a portion of the true lumen and the subintimal space.

In some aspects, the techniques described herein relate to a method, further including the step of: imaging a location for placement of the stent by intravascular ultrasound (IVUS).

In some other aspects, the techniques described herein relate to an endovascular method for bypassing an occlusion, including the steps of: (a) advancing a distal end of a guidewire through a microcatheter and into a subintimal space of an artery of a patient; (b) forming an open-loop knuckle at the distal end of the guidewire; (c) advancing the knuckle of the guidewire in the subintimal space to a location adjacent and distal to a distal end of the occlusion; (d) placing an inflatable balloon catheter over the advanced guidewire distal to the occlusion and along an entire length of the occlusion; (e) forming at least one opening in a layer separating the subintimal space from a true lumen by inflating the inflatable balloon catheter along the entire length of the occlusion; and (f) traversing the at least one opening into the true lumen with the guidewire; and wherein throughout steps c) through f), the knuckle is maintained in a position distal to a distal end of the inflatable balloon catheter.

In some aspects, the techniques described herein relate to a method, further including the step of: between steps c) and d), confirming accurate placement of the guidewire for targeted reentry by injecting a contrast media into the subintimal space.

In some aspects, the techniques described herein relate to a method, wherein: forming at least one opening of step e) further includes inflating the inflatable balloon catheter at least two times.

In some aspects, the techniques described herein relate to a method, further including the step of: after step c) and before step f), avoiding advancing the guidewire past the location of the knuckle of step c).

In some aspects, the techniques described herein relate to a method, further including the step of: after step f), inflating the inflatable balloon catheter along the length of the occlusion.

In some aspects, the techniques described herein relate to a method, further including the step of: injecting a contrast media into the true lumen antegrade the occlusion (a.k.a. contralateral injection); and injecting the contrast media into the subintimal space through the microcatheter (a.k.a Carlino's injection).

Unless otherwise defined, all technical and scientific terms used herein have the same meaning as commonly understood by one of ordinary skill in the art to which this invention belongs. The present invention may be embodied in other specific forms without departing from the spirit or essential attributes thereof, and it is therefore desired that the present embodiment be considered in all aspects as illustrative and not restrictive. Any headings utilized in the description are for convenience only and no legal or limiting effect. Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

Hereinafter, various exemplary embodiments of the disclosure are illustrated in more detail with reference to the drawings.

FIGS. 1A-1F illustrate an exemplary embodiment of an endovascular method for bypassing an occlusion, in accordance with aspects of the present disclosure.

FIG. 2 illustrates an exemplary embodiment of a vessel of a patient.

FIG. 3A illustrates an exemplary embodiment of a knuckle at a distal end of a guidewire, in accordance with aspects of the present disclosure.

FIG. 3B illustrates an exemplary knuckle at a distal end of a guidewire as contrasted with the knuckle illustrated in FIG. 3A.

FIG. 4 illustrates a representative case of the endovascular method for bypassing an occlusion, in accordance with aspects of the present disclosure.

FIG. 5 illustrates a representative case of an optical coherence tomography (OCT) image of a vessel subject to the endovascular method for bypassing an occlusion, in accordance with aspects of the present disclosure.

### DETAILED DESCRIPTION

Reference will now be made in detail to embodiments of the present disclosure, one or more drawings of which are set forth herein. Each drawing is provided by way of an explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

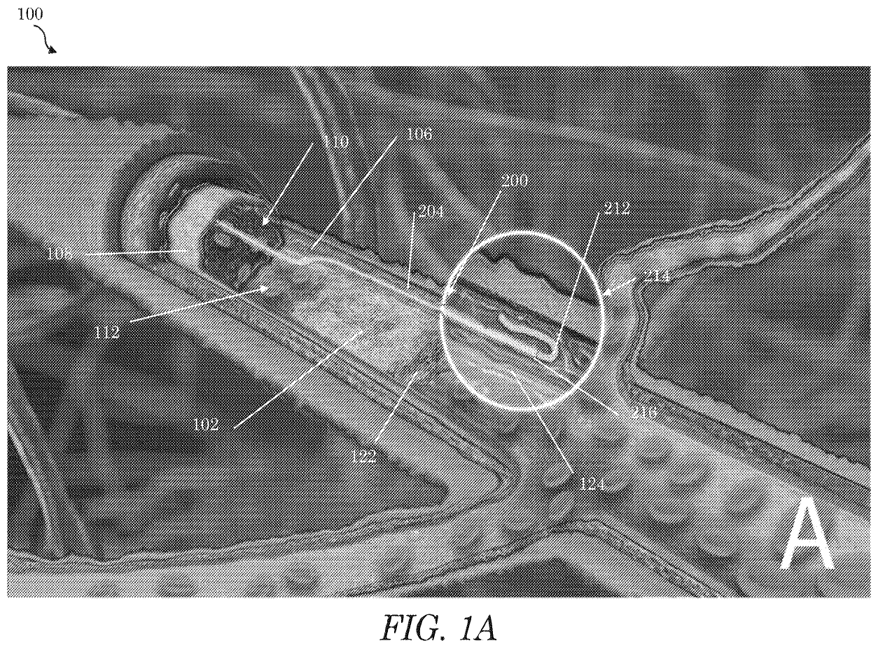

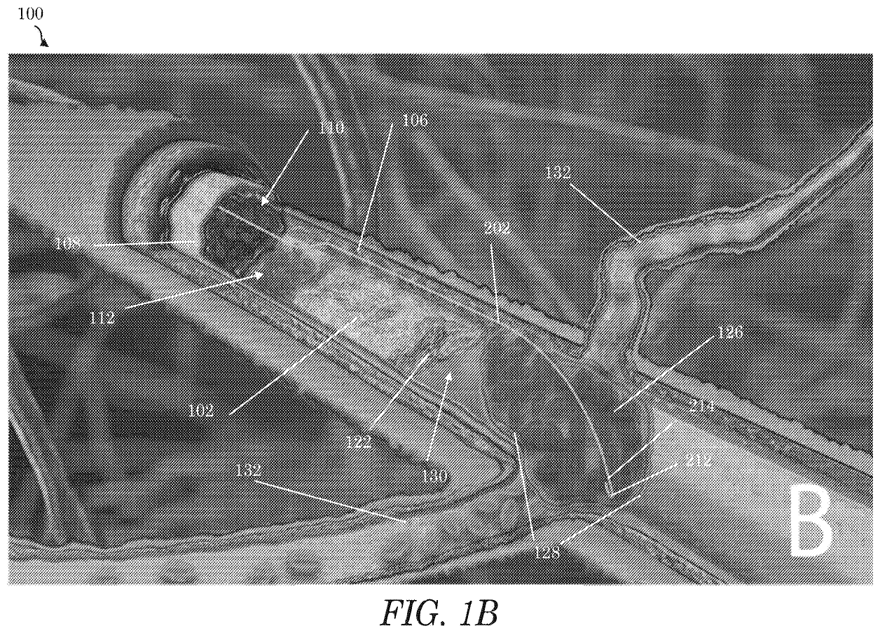

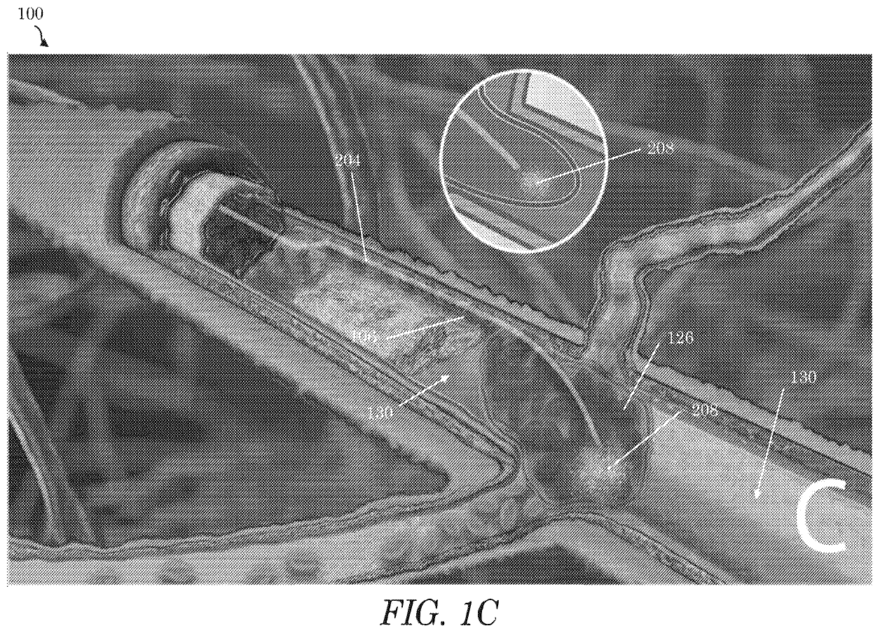

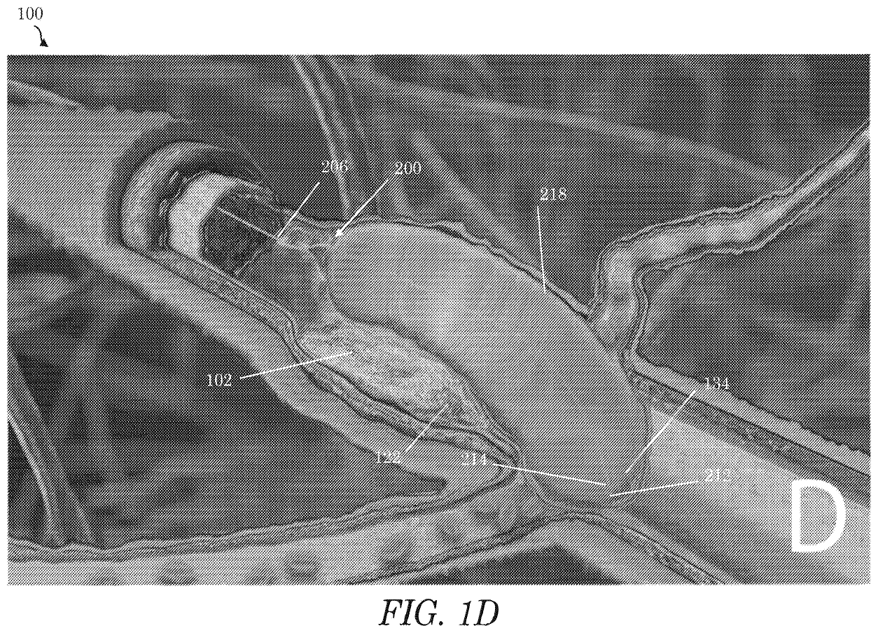

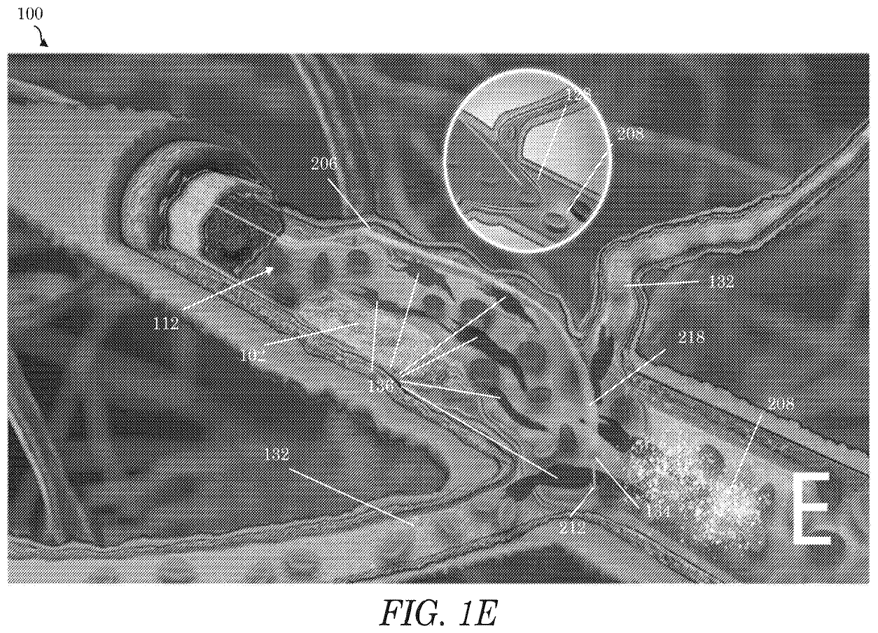

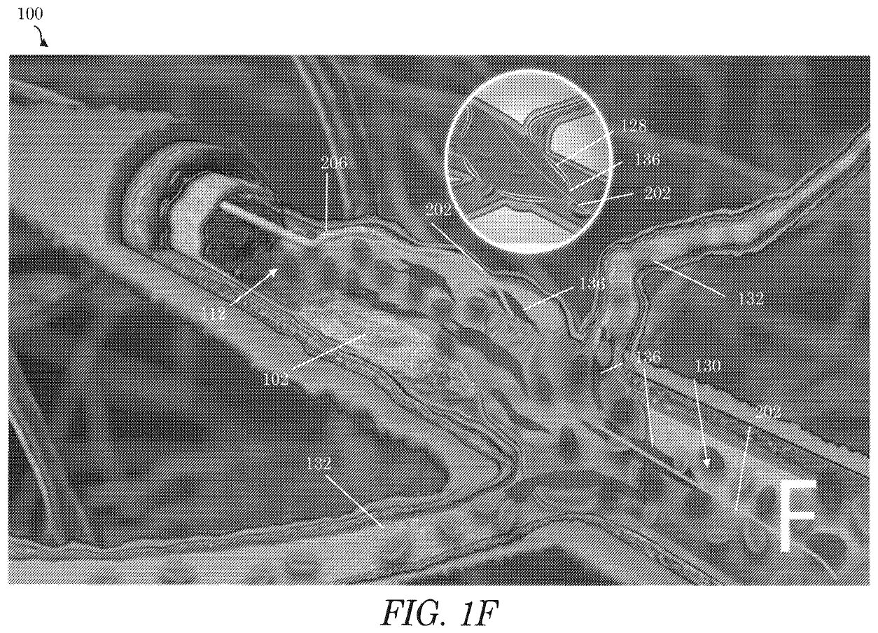

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure. Referring generally to FIGS. 1-5, various exemplary embodiments may now be described of apparatuses, systems, and methods for an endovascular method 100 for bypassing an occlusion 102. The endovascular method 100 for bypassing an occlusion may be characterized as the Subintimal Antegrade Fenestration and Reentry (SAFER) technique. Various embodiments may now described of the endovascular method 100 for bypassing an occlusion 102, and apparatuses and systems of enacting the method. Specifically, various embodiments may not be described of the endovascular method 100 for bypassing an occlusion 102, a system 200 for enacting an endovascular approach to bypass an occlusion 102, and the apparatuses included within the system 200 for enacting an endovascular approach to bypass an occlusion 102. Where the various figures describe embodiments sharing various common elements and features with other embodiments, similar elements and features are given the same reference numerals and redundant description thereof may be omitted below.

FIGS. 1A-1F illustrate an exemplary embodiment of an endovascular method 100 for bypassing an occlusion 102. The exemplary embodiment of the endovascular method 100 for bypassing an occlusion 102 may be carried out through the use of the system 200 for enacting the endovascular method 100. As illustratively conveyed in FIGS. 1A-1F, the system 200 may include a guidewire 202, a microcatheter 204, and an inflatable balloon catheter 206. In optional embodiments, the system 200 may additionally implement a contrast media 208.

Referring to FIG. 1A, the endovascular method 100 may include a step a) 104 of advancing a distal end of the guidewire 202 through the microcatheter 204 and into a subintimal space 106 of a vessel 108 of a patient. The endovascular method 100 may begin with an antegrade advancement of the guidewire 202 through the true lumen 110 of the vessel 108 in an antegrade area 112 before the occlusion 102. In the antegrade area 112, the guidewire 202 may be manipulated to enter the subintimal space 106 of the vessel 108.

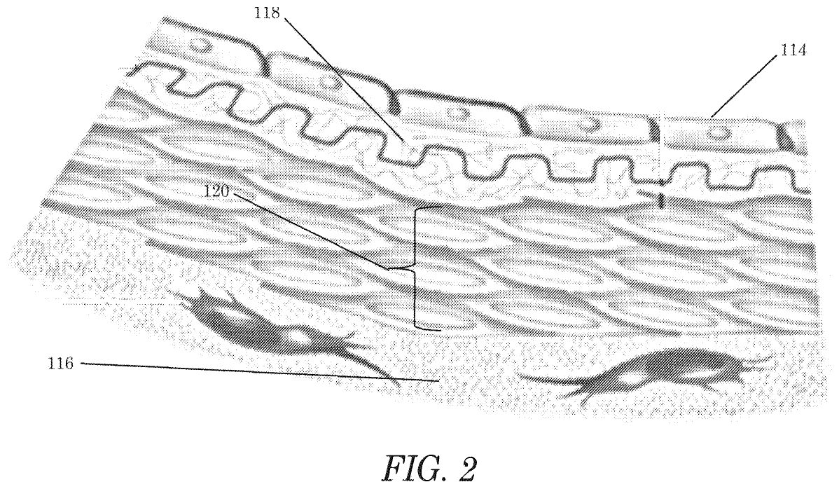

Referring to FIG. 2, reference to the subintimal space 106 may be otherwise be denoted herein as extra-plaque space and is used to refer to an area of the vessel 108 that lies between an intima 114 and adventitia 116. The vessel 108 of a patient, commonly an epicardial coronary artery, may have a wall structure that contains three main anatomical layers. An innermost layer is the intima 114, which consists of a single layer of endothelial cells supported by a thin subendothelial connective tissue layer 118 organized longitudinally. Next, a layer of fibromuscular media 120 is sandwiched between the intima 114 and the adventitia 116. The fibromuscular media 120 has a circumferential organization of nonstriated myocytes with scattered elastic membranes and a few collagen fibers. It is typically between the intima 114 and the fibromuscular media 120 that the guidewire 202 may be inserted into the subintimal space 106. The outermost layer, known as the adventitia 116, is composed of collagen and elastin fibers, which run predominantly in a longitudinal direction.

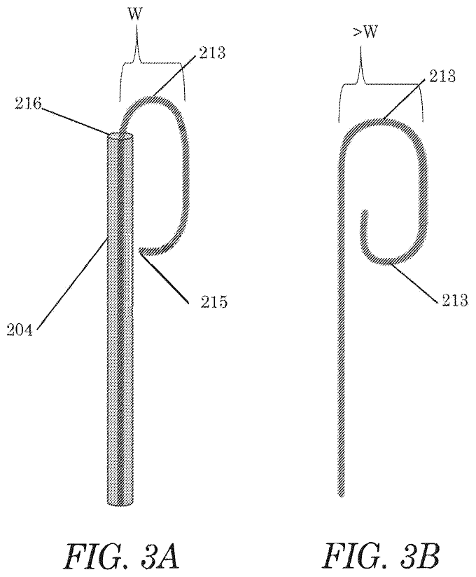

Once the guidewire 202 has entered the subintimal space 106, the endovascular method 100 may continue with a step b) of manipulating the guidewire 202 to form a knuckle 212 at a distal end 214 of the guidewire 202. In optional embodiments of the step b) of forming the knuckle 212 at the distal end 214 of the guidewire 202, the knuckle 212 may preferably be formed as a “tight” knuckle. Referring to FIG. 3A, in some aspects, the “tight” knuckle 212 may be formed as an open-loop structure, or more particularly a J-loop structure, where a distal tip 215 of the guidewire 202 does not form a closed loop with another portion of the guidewire 202. In other aspects, the “tight” knuckle 212 may be maintained as a short and small knuckled tip with a width W. The width W of the “tight” knuckle 212 may be based at least in part on the size of the vessel 108 and the specific guidewire 202. The guidewire 202 may form a tight knuckle 212 where the distal end 214 of the guidewire 202 contains only one 180-degree curved portion 213 to provide the knuckle 212. Referring to FIG. 3B, an end of a guidewire 202 is shown to contain more than one 180-degree curved portion 213, and thereby does not form a “tight” knuckle 212 within the scope of the present disclosure as it includes a width >W. An exemplary guidewire 202 capable of forming the “tight” knuckle 212 may be the ASAHI Gladius® MG guidewire or the ASAHI Gladius® Mongo® guidewire. A guidewire 202 may be selected based upon its ability to maintain a short and small knuckle 212 tip, good shaping memory, and steerability.

In optional embodiments, once the step b) of manipulating the guidewire 202 to form a knuckle 212 at a distal end 214 of the guidewire 202 is accomplished, the endovascular method 100 may continue with avoiding additional guidewire 202 manipulation related to the formation of the knuckle 212 or related to any other maneuver of the guidewire 202 other than advancing the guidewire 202 as described further herein. Referring to FIG. 1, additional guidewire 202 manipulation within the subintimal space 106 may enlarge and/or elongate a dissection plane 124 and thus lead to a hematoma expansion 126. Under systemic pressure of a circulatory system, a blood inflow is directed into the subintimal space 106 from where the guidewire 202 is advanced into the subintimal space 106. Where the guidewire 202 is manipulated beyond the step b) of manipulating the guidewire 202 to form a knuckle 212 at a distal end 214 of the guidewire 202 or otherwise manipulated in deviation from advancing the guidewire 202 as described further herein, then blood may pool in the subintimal space 106 and progressively displace a dissection flap 128 of the dissection plane 124 in longitudinal and transverse axial directions of the dissection plane 124. The progressive displacement of the dissection flap 128 of the dissection plane 124 may compress a distal true lumen 130 and may also block or otherwise exclude access to one or more side branch vessels 132, thereby preventing necessary blood flow to vessels and tissue downstream from the hematoma expansion 126.

Referring to FIG. 1A, the endovascular method 100 may continue with a step c) of advancing the guidewire 202 with the knuckle 212 at the distal end 214 to the occlusion 102. In optional embodiments in step c), the guidewire 202 with the knuckle 212 at the distal end 214 may be advanced to the occlusion 102, to a distal cap 122, or distal end, of the occlusion 102, and slightly past the distal cap 122 of the occlusion 102. The step c) of advancing the guidewire 202 with the knuckle 212 at the distal end 214 slightly past the distal cap 122 of the occlusion 102 may include advancing the knuckle 212 at least 10 mm past the distal cap 122 of the occlusion 102. In other optional embodiment in step c), the knuckle 212 at the distal end 214 of the guidewire 202 may be advanced adjacent to and distal to a distal cap 122 of the occlusion 102. The knuckle 212 at the distal end 214 of the guidewire 202 may be advanced no further distal to the occlusion 102 than is required to place a distal portion 134 of a balloon catheter 206 across the distal cap 122 of the occlusion 102. Advancing the knuckle 212 at the distal end 214 of the guidewire 202 no further distal to the occlusion 102 than is required to place a distal portion 134 of a balloon catheter 206 across the distal cap 122 of the occlusion 102 may be based at least in part on the particular balloon catheter 206 used. In an exemplary embodiment, a balloon catheter 206 with a semi-compliant balloon 218 sized 8-12 mm in length will require the knuckle 212 at the distal end 214 of the guidewire 202 to be advanced at least 10 mm past the distal cap 122 of the occlusion 102. The balloon catheter 206 may be sized according to a presumed size, or alternatively to a documented size, of the vessel 108 in which the endovascular method 100 is being performed. In optional embodiments, the balloon catheter 206 is sized for a 1:1 ratio to the presumed or documented size of the vessel 108, particularly in a location distal to the occlusion 102, in which the endovascular method 100 is being performed. In optional embodiments, the knuckle 212 at the distal end 214 of the guidewire 202 may be advanced to a position no greater than 1 mm beyond the distal cap 122 of the occlusion 102, or alternatively no greater than a range of 1 mm to 5 mm beyond the distal cap 122 of the occlusion 102, or alternatively no greater than a range of 5 mm to 15 mm beyond the distal cap 122 of the occlusion 102, or alternatively no greater than any discrete distance falling with the ranges as disclosed herein.

In optional embodiments, the step c) of advancing the guidewire 202 with the knuckle 212 at the distal end 214 to the occlusion 102 may be confirmed via a step of contralateral injection (not shown). The step of confirming advancement of the guidewire 202 to the occlusion 102 via contralateral injection (not shown) may include placing a microcatheter 204 over the guidewire 202 and subsequently withdrawing the guidewire 202 to perform a selective injection simultaneously with a contralateral coronary injection (i.e., in an occlusion distal vessel, receive blood from collaterals that can be opacified by injecting the opposite coronary vessel—a contralateral injection). Further, in embodiments utilizing contralateral injection (not shown) and embodiments that do not utilize contralateral injection (not shown), the advancement of the knuckle 212 at the distal end 214 of the guidewire 202 may be halted once the position slightly past the distal cap 122 of the occlusion 102 is reached. This position of the knuckle 212 at the distal end 214 of the guidewire 202 may be the forward most position of advancement within the endovascular method 100 and may be referred to as the “base of operations” for the step f) of advancing the guidewire 202 into the distal true lumen 130 of the vessel 108 as described further herein.

In optional embodiments, once the knuckle 212 at the distal end 214 of the guidewire 202 is advanced to the “base of operations,” the microcatheter 204 may be advanced over the guidewire 202 while maintaining the knuckle 212 at the distal end 214 of the guidewire 202 at the “base of operations.” As illustratively conveyed in FIG. 3A, the microcatheter 204 may be advanced over the guidewire 202 up to a position where a distal terminus 216 of the microcatheter 204 is positioned proximal to the knuckle 212 but distal to a portion of the guidewire 202 forming the J-loop structure. In this manner, the distal terminus 216 of the microcatheter 204 may follow the knuckle 212 to in part control the size or radius of the knuckle 212 and in part control the degree of force applied to the dissection plane 124. The microcatheter 204 may also be advanced over the guidewire 202 up to a position where a distal terminus 216 of the microcatheter 204 is positioned proximal to the knuckle 212 and in line with, or proximal to, the portion of the guidewire 202 forming the J-loop structure. In optional embodiments, once the microcatheter 204 has been advanced over the guidewire 202 while maintaining the location of the knuckle 212 at the “base of operations,” the guidewire 202 may be temporarily withdrawn.

Referring to FIG. 1C, in optional embodiments, the endovascular method 100 may include confirming the accurate placement of the guidewire 202 for targeted reentry into the true lumen 110 by injecting a contrast media 208 into the subintimal space 106. The contrast media 208 may be injected into the subintimal space 106 through the microcatheter 204. In optional embodiments, the contrast media 208 may be injected into the subintimal space 106 through the microcatheter 204 when the guidewire 202 is temporarily withdrawn from the microcatheter 204. The injection of the contrast media 208 into the subintimal space 106 may further include injecting the contrast media 208 at a location where the distal terminus 216 of the microcatheter 204 is located adjacent to the “base of operations,” or where the knuckle 212 at the distal end 214 of the guidewire 202 was maintained when the microcatheter 204 was advanced over the guidewire 202. The step of confirming accurate placement of the guidewire 202 for targeted reentry into the distal true lumen 130 by injecting a contrast media 208 into the subintimal space 106, otherwise known as a “Carlino” contrast injection, may provide for guiding placement of the balloon catheter 206 to a position where at least a distal portion 134 of the balloon catheter 206 is position to inflate across the distal cap 122 of the occlusion 102. The “Carlino” contrast injection may further provide for confirmation of at least one opening 136, otherwise known as fenestrations, as described further herein.

In optional embodiments, the endovascular method 100 may continue with advancing the guidewire 202 through the microcatheter 204 which may be located within the contrast media 208 as injected into the subintimal space 106. The presence of the contrast media 208 within the subintimal space 106 may form a contrast landmark or otherwise provide an angiographic marker for steps performed in the endovascular method 100. Once the guidewire 202 is advanced through the microcatheter 204 to a location adjacent to or at the “base of operations,” the microcatheter 204 may be removed from the subintimal space 106 and retracted from the vessel 108. Once the microcatheter 204 has been removed, the balloon catheter 206 with a semi-compliant balloon 218 may be advanced over the guidewire 202. The balloon catheter 206 and semi-compliant balloon 218 may be any suitable rapid exchange catheter and may be sized in a 1:1 ratio to the presumed or documented size of the vessel 108, or more particular the distal true lumen 130. The balloon catheter 206 with the semi-compliant balloon 218 may be advanced to a location where at least a distal portion 134 of the balloon catheter 206 is located distal to the distal cap 122 of the occlusion 102. The advancement of the balloon catheter 206 with the semi-compliant balloon 218 may be guided by the contrast media 208 that remains persistent within the subintimal space 106 and acts as a contrast landmark.

In optional embodiments, the semi-compliant balloon 218 of the balloon catheter 206 may be sized such that an entire inflatable length of the semi-compliant balloon 218 extends at least the entire length of the occlusion 102. When advancing the balloon catheter 206 with the semi-compliant balloon 218 over the guidewire 202 to a location where at least a distal portion 134 of the balloon catheter 206 is located distal to the distal cap 122 of the occlusion 102, the semi-compliant balloon 218 may be located to extend from a location spanning proximal the occlusion 102 to a location distal to the distal cap 122 of the occlusion 102 and along an entire length of the occlusion 102. The knuckle 212 may be maintained distal to distal portion 134 of the semi-compliant balloon 218 at the time of balloon inflation to facilitate the distal end 214 of the guidewire 202 to score/fracture the subintimal membrane and be projected and subsequently prolapse across the subintimal membrane into the distal true lumen 130. The relative position of the distal end 214 of the guidewire 202 and the distal portion 134 of the semi-compliant balloon 218 is illustratively conveyed in FIG. 1E.

Referring to FIG. 1D, the endovascular method 100 may continue with a step e) of inflating the balloon catheter 206 with the semi-compliant balloon 218 to form at least one opening 136 in intima 114, or layer separating the subintimal space 106 from the distal true lumen 130. The at least one opening 136 may include permanent or semi-permanent openings or fenestrations OR subintimal membrane fractures where the at least one opening 136 are not transient in nature. In optional embodiments, the semi-compliant balloon 218 may be inflated along the entire length of the occlusion 102. During inflation of the balloon catheter 206 with the semi-compliant balloon 218, the position of the guidewire 202 is maintained such that the location of the knuckle 212 is maintained at, or adjacent to, the “base of operations.” The inflation of the balloon catheter 206 with the semi-compliant balloon 218 may form more than one of the at least one opening 136, or fenestrations. In optional embodiments, the step e) of forming at least one opening 136 in the layer separating the subintimal space 106 from the distal true lumen 130 may further include deflating the balloon catheter 206 with the semi-compliant balloon 218 after an initial inflation and then inflating the balloon catheter 206 with the semi-compliant balloon 218 at least a second time. In optional embodiments, the balloon catheter 206 with the semi-compliant balloon 218 may be inflated and deflated cyclically any number of times to form the at least one opening 136 in the layer separating the subintimal space 106 from the distal true lumen 130.

Referring to FIG. 1E, the endovascular method 100 may continue with confirming the presence of the at least one opening 136 in the layer separating the subintimal space 106 from the distal true lumen 130. In optional embodiments, confirming the presence of the at least one opening 136 in the layer separating the subintimal space 106 from the distal true lumen 130 may include confirming the contrast media 208 is washed out from the subintimal space 106, particularly the hematoma expansion 126, and into the distal true lumen 130 upon the formation of the at least one opening 136, which can be demonstrated angiographically and may otherwise be known as Carlino's sign. In further embodiments, confirming the presence of the at least one opening 136 in the layer separating the subintimal space 106 from the distal true lumen 130 may include injecting a separate injection of the contrast media 208 into the true lumen 110 in a location proximal to the occlusion 102, the subintimal space 106 in a location proximal to the occlusion 102, or a combination thereof. Where the at least one opening 136 has been successfully formed and/or the occlusion 102 has been modified such that the occlusion 102 does not block an entirety of the true lumen 110, then the contrast media 208 injected into the subintimal space 106 and/or the true lumen 110 in a location proximal to the occlusion 102, respectively, will flow downstream to the distal true lumen 130 and will not aggregate within the subintimal space 106 or the true lumen 110.

Further referring to FIG. 1E, as a result of formation of at least one opening 136 in the layer separating the subintimal space 106 from the distal true lumen 130, antegrade flow to the one or more side branch vessels 132 may be restored. In optional embodiments, flow to the one or more side branch vessels 132 may be confirmed angiographically via injection of contrast media 208 as described above herein.

Referring to FIG. 1F, the endovascular method 100 may continue with a step f) of traversing the at least one opening 136 into the distal true lumen 130 with the guidewire 202. In optional embodiments, before the step f) of traversing the at least one opening 136 with the guidewire 202, the semi-compliant balloon 218 of the balloon catheter 206 may be deflated. In further optional embodiments, before the step f) of traversing the at least one opening 136 with the guidewire 202, the semi-compliant balloon 218 of the balloon catheter 206 may be deflated and may be slightly retracted from its position where the distal portion 134 of the balloon catheter 206 is located either at the position to inflate across the distal cap 122 of the occlusion 102 or at the position where the distal portion 134 of the balloon catheter 206 is located distal to the distal cap 122 of the occlusion 102. Slightly retracting the semi-compliant balloon 218 of the balloon catheter 206 may include retracting the semi-compliant balloon 218 by an amount equal to one length of the semi-compliant balloon 218, so the “base of operation” is free to provide the guidewire 202 to engaging the at least one opening 136. In an exemplary embodiment, a semi-compliant balloon 218 sized 12 mm in length is slightly retracted by retracting the semi-compliant balloon 218 by 12 mm. After the semi-compliant balloon 218 of the balloon catheter 206 has been slightly retracted, the guidewire 202 may be similarly slightly retracted. In optional embodiments, during the slight retraction of the semi-compliant balloon 218 of the balloon catheter 206 and the guidewire 202, the distal end 214 of the guidewire 202 may be maintained distal to the distal portion 134 of the balloon catheter 206. Once the semi-compliant balloon 218 of the balloon catheter 206 and the guidewire 202 have been slightly retracted, the guidewire 202 may be manipulated to traverse the at least one opening 136 and re-enter the distal true lumen 130.

In optional alternative embodiments, the step f) of traversing the at least one opening 136 into the distal true lumen 130 with the guidewire 202 may occur simultaneously with the step e) of forming at least one opening in a layer separating the subintimal space 106 from the distal true lumen 130 by inflating the inflatable balloon catheter 206. In inflating the semi-compliant balloon 218 of the balloon catheter 206, the knuckle 212 at the distal end 214 of the guidewire 202 may be propelled across the layer separating the subintimal space 106 from the distal true lumen 130, where the guidewire 202 may traverse the at least one opening 136 along with the step of inflating the semi-compliant balloon 218 of the balloon catheter 206. In optional embodiments where the guidewire 202 may traverse the at least one opening 136 upon inflation of the semi-compliant balloon 218, there is no need to retract the semi-compliant balloon 218 of the balloon catheter 206 or the guidewire 202, and the guidewire 202 may simply be advanced further into the distal true lumen 130.

In optional embodiments, throughout steps c) through f), the location of the knuckle 212 at the distal end 214 of the guidewire 202 is maintained distal to the distal portion 134 of the balloon catheter 206. Where the location of the distal portion 134 of the balloon catheter 206 is located distal to the distal cap 122 of the occlusion 102, the maintaining of the knuckle 212 at the distal end 214 of the guidewire 202 distal to the distal portion 134 of the balloon catheter 206 increases the likelihood of traversing the at least one opening 136 with the guidewire 202.

In optional embodiments, after the guidewire 202 has been manipulated to traverse the at least one opening 136 and re-enter the distal true lumen 130, the endovascular method 100 may continue with performing one or more inflations of the semi-compliant balloon 218 of the balloon catheter 206 along the length of the occlusion 102 in a position distal to proximal the occlusion 102 to prepare the vessel 108 for implantation of a stent 220.

In optional embodiments, after the guidewire 202 has been manipulated to traverse the at least one opening 136 and re-enter the distal true lumen 130, the endovascular method 100 may continue with injection the contrast media 208 into the subintimal space 106 and/or the true lumen 110 through the microcatheter 204 to confirm adequate preparation of the vessel 108. The step of confirming adequate preparation of the vessel 108 through injection of the contrast media 208 may result in disappearance of the contrast media 208 as the contrast media 208 flows from the true lumen 110 in a location proximal the occlusion 102 and or the subintimal space 106 to the distal true lumen 130. The injection of the contrast media 208 into the true lumen 110 antegrade the occlusion 102 may be a contralateral injection, and the injection of the contrast media 208 into the subintimal space 106 through the microcatheter 204 may be known as a Carlino injection. The contralateral injection and the Carlino injection may occur simultaneously or approximately simultaneously with each other to define adequate position of the distal end 214 of the guidewire 202 and the distal portion 134 of the semi-compliant balloon 218.

In optional embodiments, the endovascular method 100 may continue with fully retracting the guidewire 202 and exchanging the guidewire 202 with a workhorse wire 222 through the microcatheter 204 and delivered to a distal location in the vessel 108 suitable for placement of the stent 220. In optional embodiments, the balloon catheter 206 may be retracted after the step f) of traversing the at least one opening 136 with the guidewire 202 so that the placement of the stent 220 may occur. The placement of the stent 220 may be in a location in at least a portion of the true lumen 110 and the subintimal space 106 such that the stent bypasses a portion of the vessel 108 where the occlusion 102 is located. In other optional embodiments, the placement of the stent 220 may in a location of the true lumen 110 spanning from a location proximal the occlusion 102 to the distal true lumen 130. In optional embodiments, the step of delivering the workhorse wire 222 through the microcatheter 204 may be guided and confirmed through intravascular ultrasound (IVUS). In this manner, the endovascular method 100 may include imaging a location for placement of the stent 220 by intravascular ultrasound (IVUS).

In optional embodiments, after the balloon catheter 206 has been removed and the vessel 108 has been adequately prepared for placement of the stent 220, the decision of whether to place the stent 220 immediately, or rather delay the placement of the stent 220 to a later procedure, is left up to the discretion of an operator (not shown).

The disclosed endovascular method 100 presents several advantages over currently known antegrade dissection and re-entry (ADR) techniques, including maintaining patency in the one or more side branch vessels 132 through the area occupied by the hematoma expansion 126.

The disclosed endovascular method 100 presents several advantages over currently known antegrade fenestration and re-entry (AFR) techniques, including maintaining patency in the one or more side branch vessels 132 through the area occupied by the hematoma expansion 126, effective drainage of the subintimal hematoma, which compresses the true lumen, via the one or more inflations of the balloon within the subintimal space 106 and along the entire occlusion that will generate at least one opening, and preferably multiple tears (a.k.a permanent fenestrations), along the dissection flaps 128 separating the subintimal space 106 from the distal true lumen 130. Another key modification that separates the SAFER technique from antegrade fenestration and re-entry (AFR) techniques is the ability to provide a significant advantage of the wire re-entering the distal true lumen 130 as a byproduct of the balloon 218 action. This benefit may also be realized in part based at least on the distal end 214 of the guidewire 202 being disposed in close proximity of the distal true lumen 130. In aspects regarding the position of the crossing guidewire relative to the balloon, the antegrade fenestration and re-entry (AFR) technique maintains a wire tip proximal to a proximal end of the balloon, whereas in SAFER, the wire tip knuckle 212 is always maintained distal to the distal portion 134 of the balloon catheter 206 before, during and after balloon 218 inflations. In SAFER, the wire tip knuckle 212 effectively bypasses all the intricated dissection created within the body of the occlusion 102 and crosses directly in the distal true lumen 130. In contrast in the antegrade fenestration and re-entry (AFR) technique, the guidewire has to navigate the complex dissection/fenestration produced by the balloon actions, along the entire occlusion before reaching the distal vessel true lumen. The transient nature of the AFR fenestration make distal wire crossing more difficult. In addition, SAFER may be a single wire technique; AFR requires two (2) wires, one to deliver the balloon and one to navigate the occlusion length treated by the balloon. In SAFER, the dissecting guidewire 202 can be utilized to travel in the subintimal space 106 (EPS) and position the balloon 218, and may be further utilized also as crossing guidewire 202.

To facilitate the understanding of the embodiments described herein, a number of terms have been defined above. The terms defined herein have meanings as commonly understood by a person of ordinary skill in the areas relevant to the present invention. The terminology herein is used to describe specific embodiments of the invention, but their usage does not delimit the invention, except as set forth in the claims.

Illustrative and exemplary patient characteristics subject to the endovascular method 100 are provided below in Table 1, and angiographic and procedural characteristics associated with each patient are provided below in Table 2.

TABLE 1
Patient demographics

| | Patient #1 | Patient #2 | Patient #3 | Patient #4 | Patient #5 | Patient #6 | Patient #7 |
|---|---|---|---|---|---|---|---|
| Age | 64 | 80 | 69 | 72 | 77 | 69 | 53 |
| Gender | M | M | M | M | M | M | M |
| eGFR | 62 | 72 | 88 | 107 | 183 | 98 | 103 |
| LVEF | 60 | 50 | 60 | 50 | 50 | 55 | 55 |
| Hb | 15.6 | 12.4 | 14.2 | 13.2 | 11.1 | 12.8 | 14 |
| Diabetes | 1 | 0 | 0 | 1 | 1 | 0 | 0 |
| Hypertension | 1 | 1 | 1 | 1 | 1 | 1 | 1 |
| CCS | 2 | 3 | 2 | 2 | 2 | 2 | 1 |
| ASA | Y | Y | Y | Y | Y | Y | Y |
| Plavix | Y | Y | Y | Y | Y | Y | Y |
| Beta blocker | N | Y | Y | Y | Y | Y | Y |
| Oral DM | Y | N | N | Y | Y | N | N |
| medication | | | | | | | |
| ACEI/ARB | Y | Y | Y | Y | Y | Y | Y |
| Statin | Y | Y | Y | Y | Y | Y | N |

ACEI = angiotensin converting enzyme inhibitor; ARB = angiotensin receptor blocker; ASA = aspirin; CCS = Canadian Cardiovascular Society angina class; DM = diabetes mellitus; Hb = hemoglobin; LVEF = sleft ventricular ejection fraction; M = male; N = no; Y = yes

TABLE 2
Procedural Outcomes

| | Patient #1 | Patient #2 | Patient #3 | Patient #4 | Patient #5 | Patient #6 | Patient #7 |
|---|---|---|---|---|---|---|---|
| J-CTO score | 1 | 4 | 1 | 4 | 2 | 2 | 3 |
| Lesion length (mm) | <10 | >20 | 10-20 | >20 | <10 | <10 | >20 |
| Radiation time (min) | 30 | 53 | 32 | 51 | 42 | 38 | 36 |
| Dose (mGy) | 1228 | 2125 | 1317 | 3072 | 2820 | 2405 | 1478 |
| Contrast (cc) | 230 | 350 | 350 | 350 | 280 | 330 | 150 |
| Number of implanted stents | 2 | 4 | 2 | 5 | 3 | 1 | 2 |
| Peak post-PCI Tn (ng/ml) | 489 | 67 | 18 | 27 | 23 | 19 | 25 |

Tn = troponin
Troponin levels are expressed in nanograms/liter. Radiation time includes a combination of fluoroscopy and cine time.

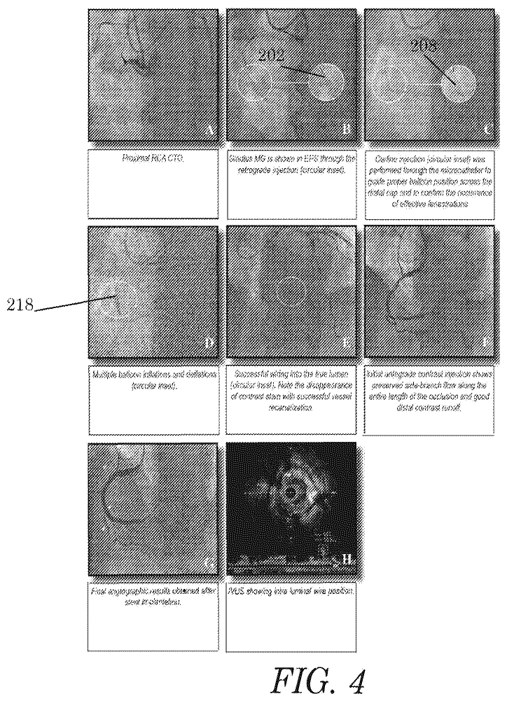

Referring to FIG. 4, a representative case of the endovascular method 100 is shown and disclosed herein. Referring to FIG. 4, panel A, a proximal right coronary artery (RCA) chronic total occlusion (CTO) is shown angiographically. Referring to FIG. 4, panel B, a Gladius® MG guidewire 202 is advanced into the subintimal space 106 as guided by a retrograde injection as shown in the magnified circle area. Referring to FIG. 4, panel C, a “Carlino” contrast injection is performed through the microcatheter 204 to guide the proper location of the balloon catheter 206 and semi-compliant balloon 218 across the distal cap 122 of the occlusion 102 and to confirm the formation of the at least one opening 136 in the layer between the subintimal space 106 and the distal true lumen 130. Referring to FIG. 4, panel D, the semi-compliant balloon 218 sized in a 1:1 ratio to the vessel 108 is inflated and deflated multiple times and positioned across the location for distal true lumen 130 re-entry and along the entire length of the occlusion 102. Referring to FIG. 4, panel E, the guidewire 202 traverses the at least one opening 136 to re-enter the distal true lumen 130. Referring further to FIG. 4, panel E, the formation of the at least one opening 136 is confirmed with the disappearance of the contrast media 208. Referring to FIG. 4, panel F, the presence of the contrast media 208 shows preserved side branch flow through the subintimal space 106 along the entire length of the occlusion 102 and adequate runoff of the contrast media 208 in the distal true lumen 130. Referring to FIG. 4, panel G, flow into the one or more side branch vessels 132 after stenting is confirmed as indicated by the arrow heads. Referring to FIG. 4, panel H, intravascular ultrasound (IVUS) is utilized to show position of the guidewire 202 within the true lumen 110 of the vessel 108.

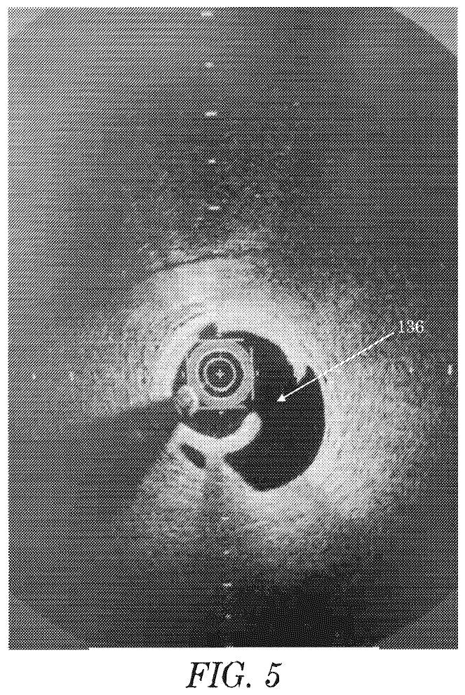

Referring to FIG. 5, an optical coherence tomography (OCT) image is provided and disclosed herein. Referring to FIG. 5, an opening of the at least one opening 136 is shown as a result of one or more inflations of the semi-compliant balloon 218 of the balloon catheter 206 (balloon size is chosen based on a 1:1 ratio between artery diameter and balloon diameter to ensure subintimal membrane fractures) to create a larger and more permanent” opening than the openings created by previous antegrade fenestration and re-entry (ADR) techniques, namely AFR. The more permanent nature of the at least one opening 136 of endovascular method 100 is shown in FIG. 5 by the discrete lesion and absence of collapsing dissection flap 128 at the side of re-entry into the distal true lumen 130.

Terms such as “a,” “an,” and “the” are not intended to refer to only a singular entity, but rather include the general class of which a specific example may be used for illustration.

The phrases “in one embodiment,” “in optional embodiment(s),” and “in an exemplary embodiment,” or variations thereof, as used herein does not necessarily refer to the same embodiment, although it may.

As used herein, the phrases “one or more,” “at least one,” “at least one of,” and “one or more of,” or variations thereof, when used with a list of items, means that different combinations of one or more of the items may be used and only one of each item in the list may be needed. For example, “one or more of” item A, item B, and item C may include, for example, without limitation, item A or item A and item B. This example also may include item A, item B, and item C, or item B and item C.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. The conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. Thus, such conditional language is not generally intended to imply that features, elements, and/or states are in any way required for one or more embodiments, whether these features, elements, and/or states are included or are to be performed in any particular embodiment.

The previous detailed description has been provided for the purposes of illustration and description. Thus, although there have been described particular embodiments of a new and useful invention, it is not intended that such references be construed as limitations upon the scope of this disclosure except as set forth in the following claims. Thus, it is seen that the apparatus of the present disclosure readily achieves the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims.

We claim:

1. An endovascular method for bypassing an occlusion, comprising the steps of:
a) advancing a distal end of a guidewire through a microcatheter and into a subintimal space of an artery of a patient;
b) forming a knuckle at the distal end of the guidewire;
c) advancing the guidewire with the knuckle at the distal end to the occlusion;
d) placing an inflatable balloon catheter over the advanced guidewire distal to the occlusion;
e) maintaining the distal end of the guidewire distal to a distal end of the balloon catheter;
f) forming at least one opening in a layer separating the subintimal space from a true lumen by inflating the inflatable balloon catheter; and
g) traversing the at least one opening with the guidewire.

2. The method of claim 1, further comprising the step of:
between steps e) and f), deflating the balloon catheter before traversing the at least one opening with the guidewire.

3. The method of claim 1, wherein step g) occurs simultaneously with step f).

4. The method of claim 1, wherein forming the knuckle of step b) further comprises the distal end of the guidewire not forming a closed loop.

5. The method of claim 1, wherein step c) further comprises advancing the knuckle no further distal to the occlusion than is required to place a distal portion of the balloon catheter across a distal end of the occlusion.

6. The method of claim 1, further comprising the step of:
between steps c) and d), confirming the accurate placement of the guidewire for targeted reentry by injecting a contrast media into the subintimal space (through a microcatheter) (near simultaneously) or after having performed a contralateral coronary injection to opacify distal lumen.

7. The method of claim 1, wherein forming the at least one opening of step e) further comprises inflating the balloon catheter at least two times.

8. The method of claim 1, wherein:
step d) further comprises placing the inflatable balloon catheter over the advanced guidewire along an entire length of the occlusion; and
forming at least one opening of step e) further comprises inflating the balloon catheter along the entire length of the occlusion.

9. The method of claim 1, further comprising the step of:
between steps e) and f), retracting the balloon catheter.

10. The method of claim 1, further comprising:
before step f), maintaining the distal end of the guidewire distal to the distal end of the balloon catheter; and
step f) further comprises forcing the distal end of the guidewire against the layer separating the subintimal space from the true lumen.

11. The method of claim 1, further comprising the step of:
after step f), injecting a contrast media into the subintimal space through the microcatheter.

12. The method of claim 1, further comprising the step of:
after step f), performing one or more inflations of the balloon catheter in a position distal to proximal of the occlusion.

13. The method of claim 1, further comprising the step of:
after step f), exchanging, through the microcatheter, the guidewire with a workhorse guidewire.

14. The method of claim 1, further comprising the step of:
after step f), removing the balloon catheter; and
placing a stent in at least a portion of the true lumen and the subintimal space.

15. The method of claim 14, further comprising the step of:
imaging a location for placement of the stent by intravascular ultrasound (IVUS).

16. An endovascular method for bypassing an occlusion, comprising the steps of:
a) advancing a distal end of a guidewire through a microcatheter and into a subintimal space of an artery of a patient;
b) forming an open-loop knuckle at the distal end of the guidewire;
c) advancing the knuckle of the guidewire in the subintimal space to a location adjacent and distal to a distal end of the occlusion;
d) placing an inflatable balloon catheter over the advanced guidewire distal to the occlusion and along an entire length of the occlusion;
e) forming at least one opening in a layer separating the subintimal space from a true lumen by inflating the inflatable balloon catheter along the entire length of the occlusion; and
f) traversing the at least one opening into the true lumen with the guidewire; and
wherein throughout steps c) through f), the knuckle is maintained in a position distal to a distal end of the inflatable balloon catheter.

17. The method of claim 16, further comprising the step of:
between steps c) and d), confirming accurate placement of the guidewire for targeted reentry by injecting a contrast media into the subintimal space.

18. The method of claim 16, further comprising the step of:
after step c) and before step f), avoiding advancing the guidewire past the location of the knuckle.

19. The method of claim 16, further comprising the step of:
after step f), inflating the inflatable balloon catheter along the length of the occlusion.

20. The method of claim 19, further comprising the step of:
injecting a contrast media into the true lumen antegrade the occlusion; and
injecting the contrast media into the subintimal space through the microcatheter.
